(19) United States
(12) Patent Application Publication
(10) Pub. No.: US 2011/0177042 A1
(43) Pub. Date: Jul. 21, 2011

(54) Method for Dedifferentiating Melanocytes

(76) Inventors: Meenhard Herlyn, Wynnewood, PA (US); Susan E. Zabierowski, Atlanta, GA (US)

(21) Appl. No.: 13/120,235

(22) PCT Filed: Sep. 30, 2009
(86) PCT No.: PCT/US09/58950

§ 371 Date: Mar. 22, 2011

Related U.S. Application Data
(60) Provisional application No. 61/102,442, filed on Oct. 3, 2008.

(51) Int. Cl. A61K 35/36; C12N 5/074; C12Q 1/02; A61K 31/713; A61K 31/19; A61P 17/00

(52) U.S. Cl. 424/93.7; 435/377; 435/29; 514/44 A; 514/557

(57) ABSTRACT

The present invention features a method for producing a dedifferentiated melanocyte. The method involves contacting a melanocyte with an agent that activates Notch1 and selecting for a dedifferentiated melanocyte which exhibits a premelanoma stem cell-like state. Agents for activating Notch1 and methods for using the cells and agents are also provided.

This application claims the benefit of priority of U.S. Provisional Application No. 61/102,442, filed Oct. 3, 2008, the content of which is incorporated herein by reference in its entirety.

This invention was supported in part by funds from the U.S. government (NCI Grant No. CA 25874, CA 076674, CA 093372 and 5 T32 CA09171) and the U.S. government has certain rights in the invention.

### INTRODUCTION

### Background of the Invention

Notch proteins are single-pass transmembrane receptors that regulate cell fate decisions during development. The Notch family includes four receptors, Notch1, Notch2, Notch3, and Notch4, whose ligands include JAG1, JAG2, DLL1, DLL3, and DLL4. All of the receptors have an extracellular domain containing multiple epidermal growth factor (EGF)-like repeats and an intracellular region containing the RAM domain, ankyrin repeats, and a C-terminal PEST domain (Das, et al. (2004) J. Biol. Chem. 279:30771-30780).

Maturation and activation of Notch1 is mediated by proteolytic processing (Chan & Jan (1998) Cell 94:423-426). A furin-like convertase within the secretory pathway cleaves Notch1 at an extracellular site, called site 1 (S1) (Logeat et al. (1998) Proc. Nat. Acad. Sci. 95:8108-8112). The resultant polypeptides associate as an intramolecular heterodimer thought to be the only form of the Notch1 receptor found on the cell surface (Logeat et al. (1998) supra). Activation of Notch1 involves cleavage between Gly1743 and Val1744 (termed site 3, or S3) (Schroeter, et al. (1998) Nature 393:382-386). S3 cleavage serves to release the Notch1 intracellular (NIC) domain from the membrane. The NIC domain then translocates to the nucleus, where it functions as a transcriptional activator in concert with CSL family members (Jarriault, et al. (1995) Nature 377:355-358). S3 processing occurs only in response to ligand binding. It has been demonstrated that ligand binding facilitates cleavage at another site, named S2, within the extracellular juxtamembrane region (Mumm, et al. (2000) Molec. Cell 5:197-206). This serves to release ectodomain repression of NIC domain production. S2 cleavage occurs between Ala1710 and Val1711, approximately 12 amino acids outside the transmembrane domain. Cleavage at S2 generates a transient intermediate peptide termed NEXT (Notch extracellular truncation). NEXT accumulates when NIC domain production is blocked by point mutations or gamma-secretase inhibitors, or by loss of presenilin-1, and inhibition of NEXT eliminates NIC domain production.

The Notch signaling network is an evolutionarily conserved intercellular signaling pathway which regulates interactions between physically adjacent cells. In Drosophilia, notch interaction with its cell-bound ligands (delta, serrate) establishes an intercellular signaling pathway that plays a key role in development. In mammals, Notch signaling has been suggested to be involved in melanocyte development, as well as maintenance of melanocyte stem cell/precursors (Moriyama, et al. (2006) J. Cell Biol. 173(3):333); however, it is not active in mature melanocytes. In addition, Notch activity has been suggested to be involved in maintenance (self-renewal), proliferation and specification of cell fate of many other adult stem cells including intestinal stem cells (Leedham, et al. (2005) J. Cell Mol. Med. 9(1):11), neural stem cells (Kageyama, et al. (2005) Exp. Cell Res. 306(2):343; Yoon & Gaiano (2005) Nat. Neurosci. 8(6):709), hematopoietic stem cell (Maillard, et al. (2005) Annu. Rev. Immunol. 23:945), mammary stem cells (Dontu, et al. (2004) Breast Cancer Res. 6:R605), muscle stem cells (Luo, et al. (2005) Semin. Cell Dev. Biol. 16:612), hair follicle stem cells (Yamamoto, et al. (2003) Curr. Biol. 13(4):333), and melanocyte stem cells.

Signaling through Notch1 is upregulated in melanoma (Balint, et al. (2005) J. Clin. Invest. 115(11):3166) and Notch1 overactivation in melanocytes has been shown to cause transformation. Moreover, aberrant expression or activity of Notch has been implicated in a variety of other cancers including T-cell acute lymphoblastic leukemia (T-ALL) (Ellisen (1991) Cell 66:649), small-cell lung cancer (Sriuranpong, et al. (2001) Cancer Res. 61:3200), neuroblastoma (Grynfeld (2000) Int. J. Cancer 88:401), cervical cancer (Zabouras, et al. (1995) Proc. Natl. Acad. Sci. USA 92:6414), prostate cancer (Shou, et al. (2001) Cancer Res. 61:7291), breast Cancer (Callahan, et al. (2004) J. Mam. Gland Biol. Neoplasia 9:145), and skin cancer (Nicolas, et al. (2003) Nat. Genetics 33(3):416).

### SUMMARY OF THE INVENTION

The present invention is a method for producing a dedifferentiated melanocyte by contacting a melanocyte with an agent that activates Notch1 and selecting for a dedifferentiated melanocyte characterized as proliferating under stem cell growth conditions, exhibiting an increase in the expression of neural crest-related genes, and exhibiting a decrease in late pigmentation-related genes as compared to melanocytes not contacted with the agent. In one embodiment, the agent is intracellular Notch1 alternatively provided as an isolated protein or a nucleic acid molecule encoding intracellular Notch1. In another embodiment, the agent is a soluble ligand of Notch1, such as Jagged1, Jagged2, Delta1 or Delta4, alternatively provided as an isolated protein or a nucleic acid molecule encoding the soluble ligand. In other embodiments, the agent is a Notch1 agonistic antibody, an inhibitory RNA molecule that blocks expression of an endogenous Notch1 inhibitor, or a small organic molecule. According to additional embodiments, the step of selecting for a dedifferentiated melanocyte comprises culturing the melanocyte in a stem cell medium. Neural crest-related genes particularly embraced by the invention include Msx1, Dlx1, Snail, Slug, Twist, p75, and SOX10, whereas late pigmentation-related genes include S100, TYRP1, MITF and HMB45. In a further embodiment, this method of the invention includes the step of differentiating the dedifferentiated melanocyte into a neuronal cell, smooth muscle cell, oligodendrocyte, melanocyte, or chondrocyte.

Dedifferentiated melanocytes, pharmaceutical compositions and tissue reconstructs are also provided, as is the use of the same in methods for promoting tissue regeneration or repair.

The present invention also features a method for identifying an agent that induces dedifferentiation of a melanocyte by contacting a melanocyte with a test agent and determining whether the test agent induces Notch1 signaling, wherein an induction in Notch1 signaling is indicative of an agent that induces dedifferentiation of a melanocyte. Agents identified by this method also find application in promoting tissue regeneration or repair.

### DETAILED DESCRIPTION OF THE INVENTION

Although previously thought to be committed to their differentiated fate, differentiated cells can be dedifferentiated. It has now been shown that forced overexpression of the active form of the stem cell regulator Notch1 in mature pigmented melanocytes activates a neural crest expression program and allows these cells to grow in stem cell media, as well as differentiate into other lineages such as osteoblasts, chondrocytes, and smooth muscle cells. Accordingly, the present invention features a method for producing a dedifferentiated melanocyte by activating Notch1 in mature, pigmented melanocytes, wherein the resulting dedifferentiated melanocytes are characterized as proliferating under stem cell growth conditions; exhibiting an increase in the expression of neural crest-related genes; and exhibiting a decrease in late pigmentation-related genes as compared to melanocytes not overexpressing intracellular Notch1. Cells produced by the method of the invention exhibit a premelanoma stem cell-like state and can be used to regenerate cells and/or tissues damaged by disease or injury.

“Differentiation” describes the acquisition or possession of one or more characteristics or functions different from that of the original cell type. A differentiated cell is one that has a different character or function from the surrounding structures or from the precursor of that cell (even the same cell). The process of differentiation gives rise from a limited set of cells (for example, in vertebrates, the three germ layers of the embryo: ectoderm, mesoderm and endoderm) to cellular diversity, creating all of the many specialized cell types that comprise an individual.

In accordance with the method of the invention, a melanocyte is contacted with an agent that activates Notch1 thereby activating a neural crest expression program. As is conventional in the art, a melanocyte is an epidermal cell capable of synthesizing melanin. Embryologically, melanocytes are derived from the neural crest, which is a different source than that of the surrounding skin cells (keratinocytes). Melanocytes are located in the bottom layer (the stratum basale) of the skin's epidermis and in the middle layer of the eye (the uvea). In the context of the present invention, a melanocyte does not include a melanoma cell. In so far as the invention contemplates in vitro and ex vivo applications, melanocytes of use in accordance with the present invention can be isolated, i.e., removed from their natural environment and other non-melanocyte cells; or alternatively be in the context of an isolated tissue or cell sample (e.g., a biopsy or skin sample).

Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway. In many, but not all tissues, the process of differentiation is coupled with exit from the cell cycle. In these cases, the cells lose or greatly restrict their capacity to proliferate and such cells are commonly referred to as being “terminally differentiated.” However, it is noted that the term “differentiation” or “differentiated” refers to cells that are more specialized in their fate or function than at a previous point in their development, and includes both cells that are terminally differentiated and cells that, although not terminally differentiated, are more specialized than at a previous point in their development.

In accordance with the present invention, “dedifferentiation” describes the process of a cell “going back” in developmental time. In this respect, a dedifferentiated cell acquires one or more characteristics previously possessed by that cell at an earlier developmental time point. An example of dedifferentiation is the temporal loss of epithelial cell characteristics during wounding and healing. Dedifferentiation can occur, in degrees. In the afore-mentioned example of wound healing, dedifferentiation progresses only slightly before the cells redifferentiate to recognizable epithelia. A cell that has greatly dedifferentiated, for example, is one that resembles a stem cell. Dedifferentiated cells can either remain dedifferentiated and proliferate as a dedifferentiated cell; redifferentiate along the same developmental pathway from which the cell had previously dedifferentiated; or redifferentiate along a developmental pathway distinct from which the cell had previously dedifferentiated. Within the context of the present invention, a dedifferentiated melanocyte is similar, but not identical, to dermis-derived neural crest-like stem cells. As such, a dedifferentiated melanocyte of the invention is multipotent, but not pluripotent.

Agents useful in practicing the method of the invention can be Notch1 agonists, which bind Notch receptor and initiate or mediate the signaling event associated with the Notch receptor; or any other agent which may or may not directly interact with Notch1 to activate Notch1 or otherwise induce the Notch1 signaling pathway. In this respect, agents that induce the expression of Notch1, increase the activity of Notch1, promote processing of Notch1 to NIC, or promote Notch1 signaling can be used to dedifferentiate melanocytes. Thus, NIC expression can be manipulated through overexpression of the corresponding nucleic acid or polypeptide, or via manipulation of, e.g., a molecule that promotes Notch1 signaling. Such agents include, but are not limited to intracellular Notch 1, soluble Notch1 ligands, anti-Notch1 agonistic antibodies, shRNA molecules that block expression of Notch1 inhibitors, and small organic molecules (e.g., a molecule having a molecular weight below about 500 Daltons) that bind the Notch receptor extracellularly or that can enter the cell and act intracellularly.

As exemplified herein, the active form of Notch1, conventionally referred to as intracellular Notch1, effectively dedifferentiates melanocytes. Activation of Notch1 involves cleavage between Gly1743 and Val1744 (Schroeter, et al. (1998) supra). This cleavage serves to release the Notch1 intracellular (NIC) domain from the membrane. Notch1 proteins can be derived from any suitable organism including human, mouse, rat, pig, and the like. In this respect, exemplary preproproteins and proproteins are described in GENBANK Accession Nos. NP_{—}001099191 (Rattus norvegicus), NP_{—}032740 (Mus musculus), NP_{—}060087 (Homo sapiens Notch1 with 19 amino acid leader sequence; Gly1743 is located at residue 1753 of this accession number), incorporated herein by reference. To produce the intracellular Notch1 domain, full-length Notch1 protein can be produced by conventional recombinant and/or chemical methodologies and subsequently cleaved between Gly1743 and Val1744. Alternatively, the NIC domain itself can be produced by conventional recombinant and/or chemical techniques. In this respect, the NIC domain is isolated in the sense that it is substantially homogeneous, e.g., as determined by SDS-PAGE under non-reducing or reducing conditions using COOMASSIE blue or, preferably, silver stain.

Isolated NIC protein can be delivered to a melanocyte, e.g., by direct contact or administration via a carrier such as a liposome or a protein transduction moiety (e.g., an HIV TAT, PTD, or Transportin peptide known to facilitate, enhance, or increase the intracellular delivery of proteins into a cell). Alternatively, the melanocyte can be contacted with a nucleic acid encoding the NIC protein. The NIC nucleic acid can be naked DNA or a vector (e.g., a plasmid or viral vector such as an adenoviral, lentiviral, retroviral, adeno-associated viral vector or the like) harboring the NIC nucleic acid. Desirably, the NIC nucleic acid provides all the necessary control sequences to facilitate expression of the NIC protein. Such expression control sequences can include but are not limited to promoter, enhancer, and polyadenylation signal sequences. Such expression control sequences, vectors and the like are well-known to those skilled in the art and routinely employed in recombinant protein expression.

Soluble Notch1 ligands are also embraced by the present invention. Natural soluble ligands for Notch1 are known in the art and include, but are not limited to Jagged1 (Lindsell, et al. (1995) Cell 80:909-917), Jagged2 (Luo, et al. (1997) Mol. Cell. Biol. 17:6057-6067), Delta1 (Heuss, et al. (2008) Proc. Natl. Acad. Sci. USA 105(32):11212-7) or Delta4 (Rao, et al. (2000) Exp. Cell Res. 260:379-86). Soluble Notch1 ligands include truncated forms of the native or natural ligands lacking the transmembrane domain, and immunoglobulin fusions of these soluble ligands where, e.g., the soluble ligand is fused to the Fc portion of an IgG. Other than the truncation, the soluble ligand can have further amino acid variations from the native sequence. These amino acid variants of the native ligand can, in the portion of the sequence that corresponds to the native sequence, have one or more amino acid changes. These amino acid changes can, e.g., confer upon the ligand, ability to constitutively activate the Notch receptor, greater binding, longer half-life and greater stability in vivo. As with NIC, a soluble notch1 receptor ligand can be synthetically or recombinantly produced or otherwise isolated; or provided to a melanocyte in the form of a nucleic acid molecule encoding the soluble ligand. Amino acid and nucleic acid molecules for human Jagged1, Jagged2, Delta1 and Delta4 are known in the art and readily available under the GENBANK Accession Nos. listed in Table 1, incorporated herein by reference.

TABLE 1

| | Nucleic Acid | Protein |
|---|---|---|
| Notch1 Ligand | Accession No. | Accession No. |
| Jagged1 | NM_000214 | NP_000205 |
| Jagged2 | NM_002226 | NP_002217 |
| | NM_145159 | NP_660142 |
| Delta1 | NM_005618 | NP_005609 |
| Delta4 | NM_019074 | NP_061947 |

In another embodiment, the present invention embraces the use of an anti-Notch1 agonistic antibody. The term “antibody” (Ab) as used herein includes monoclonal antibodies, polyclonal antibodies, multispecific antibodies (e.g., bispecific antibodies), and antibody fragments, so long as they exhibit the desired biological activity. An “isolated antibody” is one which has been identified and separated and/or recovered from a component of its natural environment. Contaminant components of its natural environment are materials which would interfere with the agonistic activity of the antibody, and may include enzymes, hormones, and other proteinaceous or nonproteinaceous solutes. Ordinarily, an isolated antibody will be prepared by at least one purification step. An “antibody fragment” includes a portion of an intact antibody, preferably the antigen binding or variable region of the intact antibody. Examples of antibody fragments include Fab, Fab′, F(ab′)₂, and Fv fragments (see, Pluckthun (1994) in The Pharmacology of Monoclonal Antibodies, vol. 113, Rosenburg and Moore eds., Springer-Verlag, New York); diabodies (see, EP 404,097; WO 93/11161; Hollinger, et al. (1993) Proc. Natl. Acad. Sci. USA, 90:6444-6448); linear antibodies (see, U.S. Pat. No. 5,641,870; Zapata, et al. (1995) Protein Eng. 8(10):1057-1062); single-chain antibody molecules; and multispecific antibodies formed from antibody fragments. In some embodiments, the antibody is humanized to contain minimal sequences derived from the non-human antibody. In general, a humanized antibody will include substantially all of at least one, and typically two, variable domains, in which all or substantially all of the hypervariable loops correspond to those of a non-human immunoglobulin and all or substantially all of the framework regions are those of a human immunoglobulin sequence. For further details, see Jones, et al. (1986) Nature 321:522-525; Riechmann, et al. (1988) Nature 332:323-329; and Presta (1992) Curr. Op. Struct. Biol. 2:593-596. Examples of anti-Notch1 agonistic antibodies are described, e.g., in U.S. Patent Application No. 20090081238 and U.S. Pat. No. 6,689,744.

Inhibitory RNA molecules, such as shRNA or siRNA are also included within the scope of the present invention. For example, siRNA that block expression of endogenous Notch1 inhibitors would be useful in activating Notch1. For example, blocking the expression of the Notch1 physiologic inhibitor Numb with siRNA designed to regions corresponding to the Numb coding sequence (5′-GCA CCU GCC CAG UGG AUC C-3′; SEQ ID NO:1) or 3′-UTR sequence (5′-GUA GCA CAU UGC AAC AAC A-3′; SEQ ID NO:2) has been shown to facilitate Notch1 recycling. See, e.g., McGill, et al. (2009) J. Biol. Chem. 284(39):26427-38). Additional inhibitory RNA molecules of use in the present method can be identified in screening assays, e.g., as described herein. As with the proteins disclosed herein, inhibitory RNA molecules can be directly provided to a melanocyte as an isolated molecule, or alternatively expressed from a recombinant expression vector.

Small organic molecules (e.g., a molecule having a molecular weight below about 500 Daltons) identified as Notch1 activators can also be used to activate Notch1 and dedifferentiate melanocytes. By way of illustration, histone deacetylase (HDAC) inhibitors valproic acid (VPA) and suberoyl bis-hydroxamic acid (SBHA) have been identified as strong Notch1 activators (Xiao, et al. (2009) Mol Cancer Ther. 8(2):350-6; Adler, et al. (2008) Surgery 144(6):956-962; Stockhausen, et al. (2005) Br. J. Cancer 92(4):751-9). As such, these agents, as well as those identified in screening assays, e.g., as described herein, find use in dedifferentiating melanocytes in the method of this invention.

Once contacted with an agent that activates Notch1, dedifferentiated melanocytes are selected for. In one embodiment, dedifferentiated melanocytes are selected for by morphological and/or phenotypical changes described herein that are associated with a dedifferentiated melanocyte. In another embodiment, dedifferentiated melanocytes are selected for by culturing the melanocytes in a stem cell medium. A stem cell medium of the present invention is a medium that facilitates the morphological and phenotypical changes associated with a dedifferentiated melanocyte. As described herein, stem cell media promotes survival, proliferation, a flattened fibroblastic phenotype, and epithelial-mesenchymal transition of a melanocyte contacted with an agent that activates Notch1. Media suitable for use in this invention include human or mouse stem cell medium, including human embryonic stem cell based medium, available from commercial sources such as Applied Stemcell, Millipore, StemCell Technologies, Inc., and Stemgent. Depending on the application of the cells, e.g., for in vitro or ex vivo manipulation of isolated cells or tissue, the medium can be solid, semi-solid or liquid.

Dedifferentiation of melanocytes can be measured by any of a number of methods including, but not limited to, assaying for a decrease in expression of one or more of the melanocyte or late pigmentation-related markers disclosed herein, assaying for an increase in proliferation or self-renewal, assaying for an increase in expression of markers of neural crest-like stem cell phenotype, and/or observing changes in cell behavior and/or morphology. According to particular embodiments, a dedifferentiated melanocyte is characterized as proliferating under stem cell growth conditions; exhibiting an increase in the expression of neural crest-related genes including, but not limited to, Msx1 (msh homeo box homolog 1), Dlx1 (distal-less homeo box 1), Snail, Slug, Twist, SOX10 (SRY-box containing gene 10) and p75 neurotrophin receptor; and exhibiting a decrease in late pigmentation-related genes including, but not limited to, MITF (microphthalmia-associated transcription factor), 5100, TYRP1 (tyrosinase-related protein 1) and HMB45, as compared to melanocytes not contacted with the agent.

The dedifferentiated cells of the invention can be used in both therapeutic applications as well as non-therapeutic applications, e.g., in studying the signaling pathways involved in differentiation and dedifferentiation, as well as in screening assay to identify therapeutic agents or molecules which facilitate dedifferentiation and redifferentiation into one or more cell lineages.

Accordingly, in one embodiment of the invention, a dedifferentiated melanocyte of the invention is redifferentiated into a cell of functional mesenchymal or neuronal lineage. In this respect, a dedifferentiated melanocyte is cultured under suitable conditions, e.g., as described herein, to induce differentiation of the dedifferentiated melanocyte into a neuronal cell, smooth muscle cell, oligodendrocyte, melanocyte, or chondrocyte. Differentiation can be monitored using any conventional marker or phenotype, which is indicative of a cell being a neuronal cell, smooth muscle cell, oligodendrocyte, melanocyte, or chondrocyte.

Relevant markers indicative of a melanocyte, dedifferentiated melanocyte, neuronal cell, smooth muscle cell, oligodendrocyte, or chondrocyte can be detected by any method available to one of skill in the art. In addition to antibodies (and antibody derivatives) that recognize and bind at least one epitope on a marker molecule, markers can be detected using analytical techniques, such as by protein dot blots, sodium dodecyl sulfate polyacrylamide gel electrophoresis (SDS-PAGE), or any other gel system that separates proteins, with subsequent visualization of the market (such as western blots), gel filtration, affinity column purification; morphologically, such as fluorescent-activated cell sorting (FACS), staining with dyes that have a specific reaction with a marker molecule (such as ruthenium red and extracellular matrix molecules), specific morphological characteristics (such as the presence of microvilli in epithelia, or the pseudopodia/filopodia in migrating cells, such as fibroblasts and mesenchyme); and biochemically, such as assaying for an enzymatic product or intermediate, or the overall composition of a cell, such as the ratio of protein to lipid. In the case of nucleic acid markers, any known method can be used. If such a marker is a nucleic acid, PCR, RT-PCR, in situ hybridization, dot blot hybridization, northern blots, Southern blots and the like can be used, coupled with suitable detection methods. If such a marker is a morphological and/or functional trait, suitable methods include visual inspection using, for example, the unaided eye, a stereomicroscope, a dissecting microscope, a confocal microscope, or an electron microscope.

Regardless of the methods of analysis, a marker, or more usually, a combination of markers, is used to identify the cells of the invention. Myofibrils, for example, are characteristic of muscle cells; axons characterize neurons, cadherins are typically expressed by epithelial cells, β2integrins are typically expressed by white blood cells of the immune system, and a high lipid content is characteristic of oligodendrocytes. These examples serve merely to illustrate the use of one or more markers to identify a particular differentiated or undifferentiated cell type.

The present invention also embraces Notch1 activators, dedifferentiated melanocytes and tissue reconstructs, and pharmaceutical compositions containing the same, for use in in vivo, in vitro, and ex vivo methods for promoting tissue regeneration and/or repair. The methods of the invention involve administering to a subject in need of treatment an effective amount of a dedifferentiated melanocyte, redifferentiated melanocyte or an agent that activates Notch1 so that tissue regeneration and/or repair is promoted. In some embodiments the method involves promoting regeneration and/or repair of skin, neurons, or muscle or bone. For example, the dedifferentiated melanocytes produced according to the method of the invention can be applied in a procedure wherein differentiated melanocytes are removed from a subject, dedifferentiated in culture, and then either reintroduced into that individual or, while still in culture, manipulated to redifferentiate along specific differentiation pathways (e.g., chondrocytes, osteogenic cells, smooth muscle, etc). Such redifferentiated cells can then be introduced to the individual. The dedifferentiated or redifferentiated melanocytes can be reintroduced at the site of injury, or can be reintroduced at a site distant from the injury. In this respect, the invention embraces autologous transplantation of dedifferentiated or redifferentiated cells (e.g., the melanocytes are harvested from and returned to the same individual). In another embodiment, the invention embraces non-autologous transplantations. In accordance with this embodiment, the transplantation occurs between a genetically related donor and recipient. In another embodiment, the transplantation occurs between a genetically un-related donor and recipient. In any of the foregoing embodiments, the invention contemplates that dedifferentiated cells can be expanded in culture and stored for later retrieval and use. Similarly, the invention contemplates that redifferentiated cells can be expanded in culture and stored for later retrieval and use.

For therapeutic applications, the cells described herein can be isolated, provided in an ex vivo tissue graft harvested from a subject, or provided in a tissue reconstruct, e.g., embedded in a synthetic matrix composed of collagen and cells (e.g., endothelial cells, fibroblasts and/or keratinocytes). The melanocytes can be contacted directly with the agent that activates Notch1 or can be transfected or transduced with a polynucleotide encoding an agent that activates Notch1.

The dedifferentiated or redifferentiated melanocytes described herein can be transplanted into an individual for the treatment of disease or injury or for gene therapy by any method known in the art which is appropriate for the type of cells being transplanted and the transplant site. For example, neural cells can be transplanted directly into the brain at the site of injury or disease.

In one example, the individual has an injury or degenerative disease, and the dedifferentiated or redifferentiated cells are reintroduced at a site of injury. When the dedifferentiated or redifferentiated cells are administered to repair cell damage due to injury and/or disease, the injury may be recent, in the process of forming scar tissue, or healed. If the injury has resulted in the formation of scar tissue or has begun to heal, the tissue may be re-injured prior to, coincident with, or subsequent to the administration of dedifferentiated or redifferentiated cells. Re-injury may help to promote regeneration resulting from administration of dedifferentiated or redifferentiated cells, however, the invention contemplates that regeneration can occur without re-injury.

Subjects benefiting from regeneration therapies include, but are not limited to, those with atherosclerosis, coronary artery disease, obstructive vascular disease, myocardial infarction, dilated cardiomyopathy, heart failure, myocardial necrosis, valvular heart disease, mitral valve prolapse, mitral valve regurgitation, mitral valve stenosis, aortic valve stenosis, and aortic valve regurgitation, carotid artery stenosis, femoral artery stenosis, stroke, claudication, and aneurysm; cancer-related conditions, such as structural defects resulting from cancer or cancer treatments; the cancers such as, but not limited to, breast, ovarian, lung, colon, prostate, skin, brain, and genitourinary cancers; skin disorders such as psoriasis; joint diseases such as degenerative joint disease, rheumatoid arthritis, arthritis, osteoarthritis, and osteoporosis; eye-related degeneration, such as cataracts, retinal and macular degenerations such as maturity onset; macular degeneration, retinitis pigmentosa, and Stargardt's disease; hearing loss; lung-related disorders, such as chronic obstructive pulmonary disease, cystic fibrosis, interstitial lung disease, emphysema; metabolic disorders, such as diabetes; genitourinary problems, such as renal failure and glomerulonephropathy; neurologic disorders, such as dementia, Alzheimer's disease, vascular dementia and stroke; and endocrine disorders, such as hypothyroidism. Finally, regeneration therapies from the methods of the invention may be very useful and beneficial for traumas to skin, bone, joints, eyes, neck, spinal column, and brain, for example, that result in injuries that would normally result in scar formation.

Using the cells and methods disclosed herein various structures in mammals can be regenerated, including skin, bone, joints, eyes (epithelium, retina, lens), lungs, heart, blood vessels and other vasculature, kidneys, pancreas, reproductive organs, tubular structures of the reproductive system (vas definers, Fallopian tubes) and nervous tissue (stroke, spinal cord injuries).

To identify additional agents useful for dedifferentiating melanocytes, the present invention also embraces a screening assay. According to this method of the invention, a melanocyte is contacted with a test agent and it is determined whether the test agent induces Notch1 signaling, wherein an induction in Notch1 signaling is indicative of an agent that induces dedifferentiation of a melanocyte. Test agents which can be screened in accordance with this assay are generally derived from libraries of agents or compounds. Such libraries can contain either collections of pure agents or collections of agent mixtures. Examples of pure agents include, but are not limited to, proteins (including polypeptides, peptides, and antibodies), nucleic acids (including DNA, RNA, siRNA, shRNA, miRNA, antisense, and ribozymes), carbohydrates, lipids, synthetic or semi-synthetic chemicals, and purified natural products. Examples of agent mixtures include, but are not limited to, extracts of prokaryotic or eukaryotic cells and tissues, as well as fermentation broths and cell or tissue culture supernates.

Library screening can be performed as disclosed herein and can be performed in any format that allows rapid preparation and processing of multiple reactions. Stock solutions of the test compounds as well as assay components can be prepared manually and all subsequent pipeting, diluting, mixing, washing, incubating, sample readout and data collecting carried out using commercially available robotic pipeting equipment, automated work stations, and analytical instruments for detecting the signal generated by the assay. Examples of such detectors include, but are not limited to, luminometers, spectrophotometers, and fluorimeters, and devices that measure the decay of radioisotopes.

After screening for an agent that activates Notch1 signaling, the compound can subsequently be tested for its ability to dedifferentiate melanocytes, e.g., as described and exemplified herein. Agents identified by this screening assay also find application in vivo for the treatment or repair of tissue.

Cells and agents described herein can be provided as is or in admixture with a pharmaceutically acceptable carrier or vehicle. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. Each carrier must be acceptable in the sense of being compatible with the other ingredients of the formulation and not injurious to the subject being treated.

The amount of cells or agents administered can depend upon a variety of factors including the nature of the cell or activity of the particular agent employed, the route of administration, the time of administration, the rate of excretion or metabolism of the particular agent being employed, the duration of the treatment, other drugs, compounds and/or materials used in combination with the particular compound employed, the age, sex, weight, condition, general health and prior medical history of the patient being treated, and like factors well known in the medical arts. A physician or veterinarian having ordinary skill in the art can readily determine and prescribe the effective amount required.

The invention is described in greater detail by the following non-limiting examples.

### Example 1

### Materials and Methods

Cells. Notch infected melanocyte spheres (FOM-NIC_MC) and GFP vector control melanocytes (FOM-GFP) were prepared as follows. FOM-NIC_MC cells, upon flow cytometry selection for GFP, initially grew as adherent cells in conventional melanocyte media (Cascade Biologicals). After 2-3 weeks, the adherent cells formed clusters of aggregating cells in the form of spheres that eventually detached and floated freely in the media. The remaining attached cells continued to form spheres. Free-floating spheres were passaged to new flasks where they attached and cells migrating out established new spheres. For culturing under different media conditions, FOM-GFP cells and FOM-NIC_MC spheres were seeded in 6-well plastic dishes for 48-72 hours in melanocyte media to establish cell attachment and proliferation. Following 72 hours, attached cells and spheres were washed with 1×PBS and replaced with HESCM4 or fresh melanocyte media (Fang, et al. (2005) Cancer Res. 65:9328-9337). Cells were incubated at 37° C., monitored daily and photographed at various intervals on a NIKON TE300 Inverted Microscope. Used medium was replaced every 4 days with fresh medium. FOM-NIC_HESCM4 spheres were cultured for at least 30 days in HESCM4 prior to use in any assay.

Differentiation Assays of NIC Infected Melanocytes. Fibroblasts and vector control melanocytes (FOM-GFP) cells were trypsinized and seeded at 20,000 cells per well in fibronectin-coated 4-well glass chamber slides in 10% DMEM and melanocyte media, respectively. Following 24 to 48 hours, cells were washed with Hanks Balanced Salt Solution (HBSS) and replaced with the appropriate differentiation media. At the same time as the medium was being replaced with differentiation media, FOM-NIC_HESCM4 spheres were collected by mechanical force, washed once with HBSS and seeded onto fibronectin-coated glass 4 well chamber slides directly into differentiation media. One half the differentiation medium was changed every other day for 14 days. Adipogenic, osteogenic, and chondrogenic media and staining were as described in the art (Fang, et al. (2005) supra). Smooth muscle differentiation medium was composed of 90% DMEM/F12 (INVITROGEN), 10% fetal bovine serum (FBS), 1% non-essential amino acids (100× stock, INVITROGEN) and 10 ng/ml TGFβ1. Alpha smooth muscle actin (SIGMA a2547) was detected after 14 days. Oligodendrocyte media was composed of 90% DMEM/F12 (3:1), 10% FBS for the first 7 days. For the next 7 days, the same medium was supplemented with 4 μM forskolin. Oligodendrocyte differentiation was assayed by staining for 2′,3′-Cyclic Nucleotide 3′-Phosphodiesterase (CNPase, CHEMICON MAB326). For re-differentiation into melanocytes, FOM-NIC_HESCM4 spheres were collected, washed with 1× phosphate-buffered saline (PBS) and seeded onto fibronectin-coated 6-well plates in melanocyte medium (Cascade Biologicals) for 3 weeks before staining cells with HMB45 and TYRP1. Images were taken using a NIKON E600 Upright microscope.

Immunofluorescent Staining and 2-Photon Imaging. FOM-GFP spheroids were generated according to known methods (Smalley, et al. (2005) Am. J. Pathol. 166:1541-1554). Briefly, 96-well plates were coated with 100 μl 1% agarose. Following agarose solidification, 5000 cells/100 μl media were seeded per well and incubated at 37° C. for 48 hours at which time the cells aggregated to form spheroids. FOM-NIC_{—}HESCM4 spheres were collected as free-floating spheres from the medium and by mechanical force if warranted. All collected spheres and spheroids were allowed to sediment, washed twice with HBSS, fixed in 4% paraformaldehyde in wash buffer (1×PBS, 0.1% TRITON X-100) for 1 hour at room temperature, blocked with 10% goat serum (1:100) for 1 hour, and incubated in primary antibody overnight at 4° C. Primary antibodies included, S100 (INVITROGEN 18-0046), HMB45 (Dako M0634), TYRP1 (Signet SIG-38150), DCT, MITF (Santa Cruz sc56726), p75, PAX3 (Zymed 38-1801), SOX10 (ABR PA1-17132), HNK1 (SIGMA c6688), and cKIT (Cell Signaling 3308). Additional primary antibodies used in this analysis included SOX2 (Novus NB100-78513), SSEA1 (Wistar), SSEA3 (Wistar), SSEA4 (Wistar), TRA-1-60 (Wistar), TRA-1-81 (Wistar) and OCT4 (Santa Cruz sc5279). Spheres were washed, and incubated with the ALEXA Fluor Series 568 secondary antibodies from INVITROGEN (all 1:600) for 1 hour at room temperature, washed repeatedly with wash buffer, and nuclei were stained using VECTASHIELD mounting media containing DAPI. Spheres were mounted on concave microscope slides and imaged on a Prairie 2-Photon Microscope. Images were complied using Velocity software.

Invasion Assay. To assess invasion, 80 μl of growth-factor reduced MATRIGEL diluted 1:6 with cold 1×PBS was added to each of CORNING 6.5 mm TRANSWELLS (8.0 μm pore size) and incubated for 30 minutes at 37° C. Fifty thousand FOM-GFP or FOM-NIC_HESCM4 cells/well were resuspended in 200 μl of 20% reduced medium and added to each of the upper chambers, while 300 μl of complete medium was added to the lower chamber. Following 24 and 48 hours of incubation at 37° C., the TRANSWELLS were fixed in 4% paraformaldehyde and washed with 1×PBS. The inner surface of the upper chamber was cleaned with a cotton swab. The membrane was removed with a scalpel and placed on a glass slide containing mounting medium with DAPI. Areas of the membranes were imaged at 10× magnification using a NIKON E600 Upright Microscope. Cells per field were calculated as an average of 25 different fields.

Migration Assay. FOM-NIC_HESCM4 spheres and FOM-GFP spheroids were implanted in a gel of bovine collagen I containing essential modified Eagle's medium, L-glutamine, and 2% fetal bovine serum according to known methods (Smalley et al. (2005) supra). Melanocyte or HESCM4 medium was overlaid on the top of the collagen. Spheroids were photographed 3 days post embedding using a TE2000 Inverted Microscope.

Human 3D Skin Reconstructs. Human in vitro 3D skin reconstructs were performed using a conventional protocol (Fukunaga-Kalabis, et al. (2006) J. Cell Biol. 175:563-569). Briefly, to each insert of tissue culture trays (Organogenesis) 3 ml of fibroblast-containing bovine type I collagen (7.5×10⁴ cells/ml) was added and allowed to constrict in DME with 10% FBS for 7 days at 37° C. FOM-NIC_HESCM4 spheres were disrupted into single cell suspension using collagenase I and IV for 30 minutes at 37° C. and strained using a 40 μm cell strainer. Dissociated FOM-NIC_HESCM4 or FOM-GFP cells (83,000 cells) were mixed with keratinocytes at a ratio of 1:5 in keratinocyte serum-free medium (INVITROGEN) containing 2% dialyzed fetal calf serum (FCS), 60 μg/ml bovine pituitary extract (INVITROGEN), 4.5 ng/ml bFGF, 100 nM human endothelin-3, and 10 ng/ml human stem cell factor (SCF). A total of 5×10⁵ cells were seeded on each contracted collagen gel. Cultures were kept submerged in medium containing 1 ng/ml EGF for 2 days, 0.2 ng/ml EGF for another 2 days, and were raised to the air-liquid interface via feeding from below with high calcium (2.4 mM) medium. After 14 days, skin reconstructs and were directly analyzed with a Prairie upright 2-photon microscope (Fukunaga-Kalabis, et al. (2006) supra).

GeneChip Expression Analysis. RNA was prepared from uninfected melanocytes (FOM), vector control melanocytes (FOM-GFP), and NIC infected melanocytes (FOM-NIC_MC and FOM-NIC_HESCM4) using QIAGEN RNEASY Mini kit. For gene expression array analysis, 150 ng RNA was reverse transcribed and labeled using an AMBION ILLUMINA TOTALPREP RNA Amplification Kit (Cat# IL1791). Hybridization was performed on ILLUMINA V2 chips and the data was analyzed via GeneSpring. Gene expression values were normalized to the mean value of all genes in each experiment.

qRT-PCR Analysis. Total mRNA was isolated using QIAGEN RNA isolation kit and 1 μg was used to synthesize cDNA using INVITROGEN SUPERSCRIPT First-Strand Synthesis System for RT-PCR, and PCR was performed using the Power SYBR Green PCR Master Mix labeling kit on an ABI 7000 Prism machine. The data was normalized to GAPDH and fold change was calculated based on melanocytes (FOM). Primer sequences were as follows: frzb forward, 5′-TGG AAG GAT CGA CTC GGT AAA-3′ (SEQ ID NO:3); frzb reverse, 5′-ACT GAG TCC AAG ATG ACG AAG CT-3′ (SEQ ID NO:4); dlx1 forward, 5′-CAG TTT GCA GTT GCA GGC TTT-3′ (SEQ ID NO:5); dlx1 reverse, 5′-TCC GGC AGA GCT AGG TAC TGA-3′ (SEQ ID NO:6); msx1 forward, 5′-ACC TCT TTG CTC CCT GAG TTC AC-3′ (SEQ ID NO:7); msx1 reverse, 5′-GAC TCT TCC AGC CAC TTT TTG G-3′ (SEQ ID NO:8), foxc1 forward, 5′-ACC CTG AAC GGA TCT ACC A-3′ (SEQ ID NO:9); foxc1 reverse, 5′-CTG CTT GTT GTC CCG GTA GAA-3′ (SEQ ID NO:10); fjx1 forward, 5′-TTC CTC GCC AAG CAC ATT TT-3′ (SEQ ID NO:11); fjx1 reverse, 5′-CCT CCC GGT GAC ACT AAG TCA-3′ (SEQ ID NO:12); snail forward, 5′-GAC TAG AGT CTG AGA TGC CC-3′ (SEQ ID NO:13), snail reverse, 5′-CAG ACA TTG TTA AAT TGC CCG-3′ (SEQ ID NO:14); twist forward, 5′-TCG AGA GAT GAT GCA GGA CGT-3′ (SEQ ID NO:15); twist reverse, 5′-TCT GGC TCT TCC TCG CTG TT-3′ (SEQ ID NO:16); fos forward, 5′-CCT CGC CCG GCT TTG-3′ (SEQ ID NO:17); fos reverse, 5′-GCC TCG TAG TCT GCG TTG AAG-3′ (SEQ ID NO:18); hes4 forward, 5′-CTC GTT AAT ACG CGC TCG-3′ (SEQ ID NO:19), hes4 reverse, 5′-AAG TCC TCC AAG CCG GTC AT-3′ (SEQ ID NO:20), hey1 forward, 5′-ACC CGA GAT CCT GCA GAT GA-3′ (SEQ ID NO:21), hey1 reverse, 5′-GCC GTA TGC AGC ATT TTC AG-3′ (SEQ ID NO:22); jag1 forward, 5′-GTG CAT GAA CGA GGT GAC CC-3′ (SEQ ID NO:23), jag1 reverse, 5′-GTA TTA ACG CCC TCG CAC GT-3′ (SEQ ID NO:24), gapdh forward, 5′-GTT CGA CAG TCA GCC GCA TC-3′ (SEQ ID NO:25), gapdh reverse, 5′-GGA ATT TGC CAT GGG TGG A-3′ (SEQ ID NO:26).

Western Blot Analysis. Whole cell extracts were isolated using RIPA buffer and 30 μg protein and analyzed by western blot using specific antibodies against Slug (Abcam ab50887), PAX3 (Zymed 38-1801), N-cadherin (BD Bioscience 610920), and E-cadherin (BD Bioscience 610182). B-actin (SIGMA a5441) was used for the purpose of loading control.

### Example 2

### Characterization of Notch1 Intracellular (NIC) Overexpressing Melanocytes

GFP or Notch1 intracellular expressing cells were either seeded directly into various media or were seeded and grown in melanocyte media for 10 days prior to replacement with, e.g., stem cell, melanocyte, melanoma and fibroblast media. The result of this analysis indicated that GFP infected melanocytes did not survive in stem cell, tumor or fibroblast media. In contrast, NIC overexpressing melanocytes cultured in melanocyte media or stem cell media survived and propagated in stem cell, melanocyte, melanoma and fibroblast media. In melanocyte medium, the NIC overexpressing cells proliferated as spheres, appearing similar to Ling's dermal spheres and embryoid bodies, whereas vector control melanocytes (FOM-GFP) cells proliferated as a monolayer similar to normal melanocytes. Moreover, NIC overexpressing melanocytes maintained Notch1 expression in both melanocyte as well as stem cell media and lost their pigment when grown in stem cell medium.

To further analyze NIC overexpressing melanocytes (FOM-NIC), the cells were grown in human embryonic stem cell based media (HESCM4). HESCM4 has been found to enrich for hair follicle stem cells (Yu, et al. (2006) Am. J. Pathol. 168:1879-1888) and melanoma stem-like cells (Fang, et al. (2005) Cancer Res. 65:9328-9337). While most GFP-infected control melanocytes cells died in HESCM4 media after 10 days, FOM-NIC spheres attached and cells emigrating from the spheres exhibited a flattened fibroblastic phenotype, in stark contrast to the typical bi-polar spindle shaped melanocytes observed in melanocyte media. Following 3 weeks in HESCM4, FOM-NIC spheres continued to survive and proliferate, generating additional detaching spheres. During this time, these additional detaching sphere cells, termed FOM-NIC_HESCM4, became unpigmented, yet continued to express comparable levels of the intracellular Notch1 transgene in comparison to FOM-NIC cells maintained in melanocyte media. Gene expression array analysis and qRT-PCR demonstrated elevated levels of the Notch ligand, Jagged1, and of Notch target genes, including HES4, HEY1, NUMBL, HES5, TNF, DTX1, MAML1, NOTCH3, NOTCH1, HES1, NUMB, DTX3, NCOR2, and NOTCH4 in FOM-NIC_HESCM4 cells compared to vector control melanocytes. While Notch components were highly expressed in FOM-NIC_HESCM4, genes involved in the melanocytic pigmentation pathway (e.g., DCHS1, TUBB3, CITED1, IL24, MITF, DDT, SILV, TYRP1, GPR143, MLPH, TYR, GPNMB, SLC45A2, DCT, and OCA2, were significantly repressed. Immunostaining and 2-Photon imaging of spheres confirmed that while the expression of DCT remained, other pigmentation indicators including MITF, TYRP1, S100 and HMB45 were lost in FOM-NIC_HESCM4 in comparison to FOM-GFP. In addition, the NIC overexpressing cells did not exhibit an increase in the expression of N-cadherin under stem cell growth conditions.

Gene expression profiling of FOM-NIC_HESCM4 also revealed the upregulation of neural crest stem cell-related genes such as MSX1, DLX1, FOXC1, DKK3, Axin2, FRZB, HOXB5, HOXB9, LHX2, ITGA5, NFKB2, CSPG4, HOXB8, FJX1, CEBPD, FOXP1, FZD4, TGFB3, BMP1, EFNA1, HOXC13, SLC29A2, DTX3, EGR1, TSC22D1, EFNB2, and FOX, a subset of which are known regulators of Wnt signaling. A more comprehensive list of genes with an increase in expression of at least 2-fold (minus background noise) in NIC overexpressing melanocytes grown in stem cell media, but not different between wild-type melanocytes and melanocytes expressing GFP is provided in Table 2. Although not apparent through array analysis, additional downstream regulators of NC cells, including Snail and Twist, as well as other NC stem cell markers, p75 and Sox10 (Wong, et al. 2006) J. Cell Biol. 175:1005-1015; Paratore, et al. (2001) Development 128:3949-3961; Takahashi, et al. (2006) Cell 126:663-676) became abundantly expressed in FOM-NIC_HESCM4.

TABLE 2

| | Fold |
|---|---|
| Gene Description | Change |
| Hairy/enhancer-of-split related with YRPW motif 2 | 12422 |
| (HEY2) | |
| cDNA FLJ43371 fis, clone NTONG2005969 | 1164 |
| guanylate cyclase 1, soluble, alpha 3 (GUCY1A3) | 689 |
| msh homeo box homolog 1 (Drosophila) (MSX1) | 366 |
| stanniocalcin 2 (STC2) | 360 |
| interferon, alpha-inducible protein 27 (IFI27) | 228 |
| cysteine-rich secretory protein LCCL domain | 181 |
| containing 2 (CRISPLD2) | |
| sulfatase 2 (SULF2), transcript variant 1 | 105 |
| sushi domain containing 4 (SUSD4) | 73 |
| distal-less homeo box 1 (DLX1) | 68 |
| protein phosphatase 1, regulatory (inhibitor) | 61 |
| subunit 14A (PPP1R14A) | |
| tripartite motif-containing 22 (TRIM22) | 60 |
| hairy/enhancer-of-split related with YRPW motif- | 56 |
| like (HEYL) | |
| collagen, type VIII, alpha 1 (COL8A1), transcript | 51 |
| variant 2 | |
| chemokine (C-C motif) ligand 2 (CCL2) | 45 |
| tropomyosin 2 (beta) (TPM2), transcript variant 1 | 44 |
| ATH1, acid trehalase-like 1 (yeast) (ATHL1) | 40 |
| lumican (LUM) | 37 |
| FBJ murine osteosarcoma viral oncogene homolog B | 34 |
| (FOSB) | |
| forkhead box C1 (FOXC1) | 28 |
| jagged 1 (Alagille syndrome) (JAG1) | 26 |
| 2′,5′-oligoadenylate synthetase 1, 40/46 kDa | 23 |
| (OAS1), transcript variant 2 | |
| tropomyosin 2 (beta) (TPM2), transcript variant 2 | 23 |
| major histocompatibility complex, class I, F | 22 |
| (HLA-F) | |
| serpin peptidase inhibitor, clade I | 21 |
| (neuroserpin), member 1 (SERPINI1) | |
| solute carrier family 45, member 4, transcript | 20 |
| variant 4 (SLC45A4) | |
| hypothetical protein FLJ37440 (FLJ37440) | 19 |
| interferon regulatory factor 1 (IRF1) | 19 |
| glycoprotein Ib (platelet), beta polypeptide | 14 |
| (GP1BB) | |
| spondin 2, extracellular matrix protein (SPON2) | 14 |
| transforming growth factor, beta-induced, 68 kDa | 13 |
| (TGFBI) | |
| phosphatidic acid phosphatase type 2A (PPAP2A), | 13 |
| transcript variant 2 | |
| cDNA FLJ34755 fis, clone NHNPC1000034 | 13 |
| hypothetical LOC554223, transcript variant 3 | 13 |
| (LOC554223) | |
| phosphatidic acid phosphatase type 2A (PPAP2A), | 12 |
| transcript variant 1 | |
| caldesmon 1 (CALD1), transcript variant 1 | 12 |
| hairy and enhancer of split 4 (Drosophila) (HES4) | 12 |
| tripartite motif-containing 9 (TRIM9), transcript | 11 |
| variant 1 | |
| chromosome 22 open reading frame 8 (C22orf8) | 11 |
| v-fos FBJ murine osteosarcoma viral oncogene | 11 |
| homolog (FOS) | |
| protein tyrosine phosphatase type IVA, member 3, | 11 |
| transcript variant 3 (PTP4A3) | |
| nuclear receptor coactivator 7 (NCOA7) | 10 |
| protein tyrosine phosphatase type IVA, member 3 | 10 |
| (PTP4A3), transcript variant 2 | |
| Rho guanine nucleotide exchange factor (GEF) 17 | 10 |
| (ARHGEF17) | |
| discoidin domain receptor family, member 1 | 10 |
| (DDR1), transcript variant 2 | |
| kynureninase (L-kynurenine hydrolase) (KYNU), | 10 |
| transcript variant 1 | |
| metallothionein 1F (functional) (MT1F) | 10 |
| olfactomedin-like 2A (OLFML2A) | 10 |
| hypothetical protein LOC283537 (LOC283537) | 10 |
| myosin, light polypeptide kinase (MYLK), | 9 |
| transcript variant 6 | |
| tenascin C (hexabrachion) (TNC) | 9 |
| solute carrier family 2 (facilitated glucose | 9 |
| transporter), member 1 (SLC2A1) | |
| transporter 2, ATP-binding cassette, sub-family B | 9 |
| (MDR/TAP) (TAP2), transcript variant 1 | |
| immunoglobulin superfamily, member 4B (IGSF4B) | 9 |
| laminin, alpha 5 (LAMA5) | 9 |
| tripartite motif-containing 5 (TRIM5), transcript | 8 |
| variant alpha | |
| inositol 1,4,5-triphosphate receptor, type 2 | 8 |
| (ITPR2) | |
| protease, serine, 23 (PRSS23) | 8 |
| TNFAIP3 interacting protein 1 (TNIP1) | 8 |
| cingulin (CGN) | 8 |
| La ribonucleoprotein domain family, member 6 | 8 |
| (LARP6), transcript variant 2 | |
| PTK7 protein tyrosine kinase 7 (PTK7), transcript | 8 |
| variant PTK7-4 | |
| ERBB receptor feedback inhibitor 1 (ERRFI1) | 8 |
| histone 2, H2aa (HIST2H2AA) | 8 |
| nuclear factor of kappa light polypeptide gene | 7 |
| enhancer in B-cells inhibitor, epsilon (NFKBIE) | |
| ADAM metallopeptidase with thrombospondin type 1 | 7 |
| motif, 1 (ADAMTS1) | |
| vasohibin 1 (VASH1) | 7 |
| sushi domain containing 1 (SUSD1) | 7 |
| interferon regulatory factor 7 (IRF7), transcript | 7 |
| variant c | |
| C1q and tumor necrosis factor related protein 6 | 7 |
| (C1QTNF6), transcript variant 1 | |
| glutathione peroxidase 7 (GPX7) | 7 |
| bone morphogenetic protein 1 (BMP1), transcript | 7 |
| variant BMP1-3 | |
| transmembrane protein 45A (TMEM45A) | 7 |
| TAP binding protein (tapasin) (TAPBP), transcript | 7 |
| variant 1 | |
| chromosome 20 open reading frame 35 (C20orf35), | 7 |
| transcript variant 1 | |
| 2′-5′-oligoadenylate synthetase 3, 100 kDa (OAS3) | 7 |
| zinc fingers and homeoboxes 2 (ZHX2) | 7 |
| solute carrier family 2 (facilitated glucose | 7 |
| transporter), member 10 (SLC2A10) | |
| nuclear factor of kappa light polypeptide gene | 7 |
| enhancer in B-cells 2 (p49/p100) (NFKB2) | |
| butyrophilin, subfamily 3, member A1 (BTN3A1), | 7 |
| transcript variant 1 | |
| BH3 interacting domain death agonist (BID), | 7 |
| transcript variant 2 | |
| collagen, type V, alpha 2 (COL5A2) | 6 |
| heparin-binding EGF-like growth factor (HBEGF) | 6 |
| 2′-5′-oligoadenylate synthetase 2, 69/71 kDa | 6 |
| (OAS2), transcript variant 3 | |
| KIAA0247 (KIAA0247) | 6 |
| KIAA0922 (KIAA0922) | 6 |
| hypothetical LOC440160 (LOC440160) | 6 |
| adenosine deaminase (ADA) | 6 |
| cDNA FLJ38512 fis, clone HCHON2000503 | 6 |
| sperm equatorial segment protein 1 (SPESP1) | 6 |
| legumain (LGMN), transcript variant 1 | 6 |
| serine dehydratase-like (SDSL) | 6 |
| sema domain, seven thrombospondin repeats (type 1 | 6 |
| and type 1-like), transmembrane domain (TM) | |
| La ribonucleoprotein domain family, member 6 | 6 |
| (LARP6), transcript variant 1 | |
| family with sequence similarity 101, member B | 6 |
| (FAM101B) | |
| cytoskeleton-associated protein 4 (CKAP4) | 6 |
| poly (ADP-ribose) polymerase family, member 12 | 6 |
| (PARP12) | |
| N-myc (and STAT) interactor (NMI) | 6 |
| phosphatidylserine decarboxylase (PISD) | 6 |
| tripartite motif-containing 5 (TRIM5), transcript | 6 |
| variant gamma | |
| interferon, alpha-inducible protein (clone IFI- | 6 |
| 15K) (G1P2) | |
| squamous cell carcinoma antigen recognized by T | 6 |
| cells 2 (SART2) | |
| related RAS viral (r-ras) oncogene homolog (RRAS) | 6 |
| caldesmon 1 (CALD1), transcript variant 2 | 6 |
| hairy and enhancer of split 5 (Drosophila) (HES5) | NA |
| jagged 1 (Alagille syndrome) (JAG1) | NA |
| tumor necrosis factor (TNF superfamily, member 2) | NA |
| (TNF) | |
| Notch homolog 3 (Drosophila) (NOTCH3) | NA |
| deltex homolog 1 (Drosophila) (DTX1) | NA |
| Notch homolog 4 (Drosophila) (NOTCH4) | NA |
| deltex 3 homolog (Drosophila) (DTX3) | NA |
| Notch homolog 1, translocation-associated | NA |
| (Drosophila) (NOTCH1) | |
| numb homolog (Drosophila) (NUMB), transcript | NA |
| variant 3 | |
| numb homolog (Drosophila)-like (NUMBL) | NA |
| hairy and enhancer of split 1, (Drosophila) | NA |
| (HES1) | |
| recombining binding protein suppressor of | NA |
| hairless (Drosophila) (RBPSUH), transcript | |
| variant 4 | |
| deltex homolog 2 (Drosophila) (DTX2) | NA |
| delta-like 1 (Drosophila) (DLL1) | NA |
| nuclear receptor co-repressor 2 (NCOR2) | NA |
| GCN5 general control of amino-acid synthesis 5- | NA |
| like 2 (yeast) (GCN5L2) | |
| presenilin 1 (Alzheimer disease 3) (PSEN1), | NA |
| transcript variant I-374 | |
| delta-like 4 (Drosophila) (DLL4) | NA |
| mastermind-like 1 (Drosophila) (MAML1) | NA |
| ADAM metallopeptidase domain 17 (tumor necrosis | NA |
| factor, alpha, converting enzyme) (ADAM17) | |
| Notch homolog 2 (Drosophila) (NOTCH2 | NA |
| radical fringe homolog (Drosophila) (RFNG) | NA |
| CBF1 interacting corepressor (CIR), transcript | NA |
| variant 1 | |
| deltex homolog 2 (Drosophila) (DTX2) | NA |
| dishevelled, dsh homolog 1 (Drosophila) (DVL1), | NA |
| transcript variant 1 | |
| skeletal muscle and kidney enriched inositol | NA |
| phosphatase (SKIP), transcript variant 2 | |
| cDNA clone IMAGE: 1421770 | NA |
| presenilin 1 (Alzheimer disease 3) (PSEN1), | NA |
| transcript variant I-467 | |
| SPHK1 (sphingosine kinase type 1) interacting | NA |
| protein (SKIP) | |
| dishevelled, dsh homolog 1 (Drosophila) (DVL1), | NA |
| transcript variant 3 | |
| deltex 4 homolog (Drosophila) (DTX4) | NA |
| potassium inwardly-rectifying channel, subfamily | NA |
| J, member 5 (KCNJ5) | |
| recombining binding protein suppressor of | NA |
| hairless (Drosophila) (RBPSUH), transcript | |
| variant 1 | |
| C-terminal binding protein 2 (CTBP2), transcript | NA |
| variant 2 | |
| BX111393 Soares fetal liver spleen 1NFLS Homo | NA |
| sapiens cDNA clone IMAGp998O14113 | |
| Notch homolog 2 (Drosophila), transcript variant | NA |
| 2 (NOTCH2) | |
| pre T-cell antigen receptor alpha (PTCRA) | NA |
| cDNA clone IMAGE: 6618132 5 | NA |
| manic fringe homolog (Drosophila) (MFNG) | NA |
| cDNA clone IMAGE: 5295478 | NA |
| similar to cytoplasmic beta-actin (LOC643897) | NA |
| recombining binding protein suppressor of | NA |
| hairless (Drosophila)-like (RBPSUHL) | |
| presenilin 1 (Alzheimer disease 3) (PSEN1), | NA |
| transcript variant I-463 | |
| 17000600014402 GRN_PREHEP Homo sapiens cDNA 5 | NA |
| mastermind-like 1 (Drosophila) (MAML1) | NA |
| C-terminal binding protein 1 (CTBP1), transcript | NA |
| variant 2 | |
| chromosome 21 open reading frame 33 (C21orf33), | NA |
| nuclear gene encoding mitochondrial protein | |
| SNW domain containing 1 (SNW1) | NA |
| mastermind-like 3 (Drosophila) (MAML3) | NA |
| lunatic fringe homolog (Drosophila) (LFNG) | NA |
| C-terminal binding protein 2 (CTBP2), transcript | NA |
| variant 1 | |
| pleckstrin homology domain containing, family M | NA |
| (with RUN domain) member 2 | |
| anterior pharynx defective 1 homolog B (C. | NA |
| elegans) (APH1B) | |
| nicastrin (NCSTN) | NA |
| radixin (RDX) | NA |
| radical fringe homolog (Drosophila) (RFNG) | NA |
| histone deacetylase 1 (HDAC1) | NA |
| histone deacetylase 2 (HDAC2) | NA |
| CREB binding protein (Rubinstein-Taybi syndrome) | NA |
| (CREBBP) | |
| jagged 2 (JAG2), transcript variant 1 | NA |
| dishevelled, dsh homolog 2 (Drosophila) (DVL2) | NA |
| dishevelled, dsh homolog 3 (Drosophila) (DVL3) | NA |
| anterior pharynx defective 1 homolog A (C. | NA |
| elegans) (APH1A) | |
| presenilin 2 (Alzheimer disease 4) (PSEN2), | NA |
| transcript variant 2 | |
| p300/CBP-associated factor (PCAF) | NA |
| delta-like 3 (Drosophila) (DLL3), transcript | NA |
| variant 1, | |

These data indicated that melanocytes overexpressing Notch could survive and proliferate in stem cell media while downregulating pigmentation pathway genes and turning on genes involved in neural crest stem cells. This indicated that, under stem cell culture conditions, Notch was activating a de-differentiation program in melanocytes toward NC precursors. However, a complete reprogramming to an induced pluripotent state (iPS) was not achieved, as these cells remained negative for the embryonic stem cell markers SSEA3, SSEA4, TRA-1-60, TRA-1-81, and OCT4.

To demonstrate the stem cell-like nature of the NIC overexpressing melanocytes, the cells were cultured under various growth conditions to induce differentiation into different mesenchymal lineages. Specifically, normal fibroblasts, vector control melanocytes (FOM-GFP) or NIC overexpressing melanocytes grown in melanocyte media or stem cell media were subjected to osteoblast, adipogenic, condrogenic, oligodendric, or smooth muscle cell inducing medium for 14 days and stained for the appropriate markers (e.g., alkaline phosphatase for osteoblast differentiation, Oil Red O stain and hematoxolysin counterstaining for adipocyte differentiation, collagen II staining for chondrocyte differentiation, CNPase staining for oligodendrocyte differentiation, and alpha smooth muscle actin staining for smooth muscle cell differentiation). This analysis indicated that NIC overexpressing melanocytes could differentiate into osteoblasts, chondrocytes, oligodendrocytes, and smooth muscle cells, but could not differentiate into adipocytes. In addition, smooth muscle-differentiated FOM-NIC_HESCM4 cells were functionally active as they were capable of constricting collagen I, unlike smooth muscle differentiated FOM-GFP cells. FOM-NIC_HESCM4 cells could also re-differentiate into pigmented, bi-polar spindle shaped melanocytes expressing melanocyte markers TYRP1 and HMB45 when grown in the appropriate medium. These data indicate that like primitive neural crest cells, FOM-NIC_HESCM4 cells could differentiate into multiple lineages.

The overt morphological change observed when FOM-NIC's were switched from melanocyte to stem cell media, from bi-polar spindle shaped cells toward fibroblastic-like cells was reminiscent of epithelial-mescenchymal transition (EMT). NC stem cells residing within the neural tube are known to undergo EMT resulting in cytoskeletal and morphological changes that promote delamination and migration away from the neural tube (Sauka-Spengler, et al. 2008) Nat. Rev. Mol. Cell. Biol. 9:557-568). Protein expression and qRT-PCR demonstrated that EMT indicators, SLUG and SNAIL1, which are also direct targets of Notch signaling (Leong, et al. (2007) J. Exp. Med. 204:2935-2948), were highly upregulated in FOM-NIC_HESCM4 cells. Additionally, E-cadherin expression, known to be repressed by snail/slug members during EMT (Cano, et al. (2000) Nat. Cell Biol. 2:76-83), was attenuated. These data indicated that under stem cell culture conditions, FOM-NIC cells had undergone EMT. It was therefore determined whether these cells had also gained increased motility. Boyden chamber migration assays demonstrated that FOM-NIC_HESCM4 possessed increased invasive capacity through MATRIGEL® compared to GFP-infected controls. When embedded into a semi-solid matrix of collagen I, FOM-NIC_HESCM4 cells also demonstrated enhanced migratory capacity compared to FOM-GFP. Moreover, when substituted for FOM-GFP in three dimensional (3D) human skin reconstructs in which melanocytes normally home to and remain at the basal layer, FOM-NIC_HESCM4 cells were extremely invasive into the fibroblast-rich dermal portion. These data indicated that FOM-NIC cells cultured under stem cell conditions lose melanocytic properties while upregulating Slug and downregulating E-cadherin, indicating that these cells had undergone EMT to become migratory NC-like cells. In addition, colony forming efficiency in soft agar was determined. This analysis demonstrated that NIC overexpressing melanocytes could efficiently form colonies from single cells when grown in stem cell soft agar medium, but not melanocyte soft agar growth medium. Control melanocytes expressing GFP likewise failed to form colonies in soft agar. However, xenotransplantation into NOD/SCID mice failed to generate tumors even after 5 months or when additional oncogenes such as BRAFV600E, NRASQ61R or CDK4 were overexpressed or tumor suppressor genes such as p53, p16INK4A or PTEN were downregulated with shRNA. Therefore, these results indicate that the cells are non-tumorigenic.

Collectively, these data demonstrate that FOM-NIC cells have reactivated primitive neural crest genes and gained biological attributes of neural crest cells. Moreover, these cells maintain viability and retain the capacity to differentiate indefinitely into multiple functional mesenchymal and neuronal lineages without giving rise to tumors in NOD/SCIDs. This indicates that otherwise mature melanocytes can undergo partial reprogramming to multipotent neural crest cells solely through the activation of Notch1 signaling.

### Example 3

### Use of Natural Ligands to Activate Notch1

It is expected that natural ligands for Notch activation, particularly Jagged1 and Delta1, are expressed by keratinocytes and can induce de-differentiation to stem cells. Notch ligands expressed on juxtaposed cells are candidates for de-differentiation because they are both important in skin and melanocyte differentiation.

To demonstrate the use of soluble ligands for Notch activation, soluble recombinant Jagged1 and Delta1 proteins are produced by constructing baculovirus vectors, wherein the extracellular domain of Jagged1/Delta1 is linked to the Fc fragment of an antibody. The proteins are expected to be readily secreted by the insect cells and be bioactive (Smith, et al. (1983) Mol. Cell. Biol. 3:2156-2165). They can be purified through a myc tag detectable by antibodies. Alternatively, recombinant Jagged1 and Delta1 proteins can be purchased from R&D Systems (Minneapolis, Minn.). Melanocytes are then incubated with the soluble protein adsorbed to plastic binding polyclonal anti-Fc antibodies and assessed for de-differentiation. As an alternative to incubation of melanocytes with soluble protein, the full length Jagged1 and Delta1 proteins can be overexpressed in keratinocytes, such that co-culturing of these keratinocytes with naïve melanocytes will activate Notch-mediated dedifferentiation via cell-cell communication and reprogram the melanocytes to multi-potent neural crest-like stem cells. Parameters for assessing de-differentiation include a decrease in TYRP-1 expression as a marker of pigmentation; and an increase in p75NGFR, Slug, Snail and Twist expression as markers for neural crest stem cells.

In skin reconstructs (skin equivalents or synthetic skin), keratinocytes are exposed to air, which triggers their differentiation into multiple layers in the same way as in natural skin. The complex epidermal layer sits on a layer of collagen with embedded fibroblasts, in which the fibroblasts control stiffness. To induce de-differentiation when melanocytes are maintained in a skin-equivalent model, in which they are dispersed among basal layer keratinocytes, Jagged1 or Delta1 are expressed under an inducible promoter in the keratinocytes. The inducible vector allows for the establishment of an epidermis of ‘normal’ keratinocytes prior to triggering activation of Notch by inducing Jagged/Delta. Because melanocytes can receive signals from fibroblasts even when separated by a basement membrane, Jagged1 and Delta1 can also be expressed by these cells for activation of Notch signaling in the melanocytes.

From these experiments, it is expected that soluble Jagged1 or Delta1 will de-differentiate melanocytes when in monolayer culture, as well as when cells are embedded in the synthetic epidermis. The challenge is to de-differentiate melanocytes under stem cell conditions while at the same time maintaining vigorous growth of the keratinocytes to allow the formation of a multi-layered epidermis. Therefore, if keratinocytes do not survive under stem cell growth conditions, then the inducible vector system can be used as this would allow skin reconstruction prior to triggering Notch activation through Jagged1. For controls, a dominant-negative mutant to Mastermind1 (MAML1) can be used to block Notch activity; γ-secretase inhibitors could also be exploited to block Notch activation. In the normal microenvironment, there is a possibility that the differentiated melanocytes, which reside in human skin, may have mechanisms to downregulate Notch signaling such as inhibition of γ-secretase or ADAM/TACE family. As alternative strategies, these proteases can be expressed in melanocytes in combination with the keratinocytes which overexpress Notch ligands.

### Example 4

### Small Molecule Screen for Notch1 Activators

To induce committed stem cells, not pluripotent cells, it is expected that chemical compounds which trigger one or more genes can be used to reprogram cells (Huangfu, et al. (2008) Nat. Biotechnol. 26:795-797; Shi, et al. (2008) Cell Stem Cell 3:568-574). To screen for such compounds, a lentiviral vector that will express luciferase only under conditions of Notch activation is constructed. The vector includes the luciferase gene, the expression of which is driven by NIC binding to a concatemer (×8) of CSL (CBF-1/Suppressor of Hairless/Lag-1) consensus binding sequences. After stable infection of TYRP-1 positive melanocytes with the luciferase vector, luciferase expression is correlated with Notch activation after exogenous stimulation by small molecules, wherein a relative increase in luciferase expression is indicative of a compound that activates Notch1 signaling.

Screening of compounds in this manner can be carried out in a 96-well format for small libraries, or in 384-well plates to test as many as 14,400 compounds (e.g., the Maybridge Hitfinder collection). Generally, libraries are formatted for screening in 96 and 384 well plates with sufficient reserves for ‘cherry-picking’ hits from primary screens for subsequent confirmation studies. Although dependent on the individual molecular composition of each compound and final assay volume, it is expected that the effective screening concentration of each compound will range from 5-25 μM. After counterscreening with a non-relevant negative control vector (e.g., a vector containing a non-specific scrambled sequence instead of the CSL concatemer), all ‘hits’ are individually confirmed and cells are analyzed for activation of Notch1 through immunostaining. These cells can then be tested for expression of Sox2 as a marker for neural crest stem cells. For controls, Oct4 expression can be analyzed to determine if the cells have de-differentiated to an embryonic stem-like cell; and TYRP-1 expression should be decreased.

In addition to small organic chemical libraries, libraries of shRNA can be screened. Such libraries can be infected into melanocytes in 50-clone pools. Screening is done as for the chemical compounds and the readout is increased luciferase activity with validation through increases in Sox2 expression. If an increase in expression of Notch1 activity is problematic, the strategy can be revised to screen for induction of expression of Sox2 or relevant markers.

Once changes in cells related to differentiation with selected compounds have been validated, it is expected that the selected compounds can be combined with the soluble ligands. In this respect, gene transduction can be combined with small molecules. Many of these experiments are expected to progress in an empirically progressive way, in which the results of one step will prepare for the next.

### Example 5

### Grafting of Skin Reconstructs

In addition to 3D reconstructs, in vivo skin reconstruct grafts are used to demonstrate efficacy. Grafting human skin reconstructs to an immunocompromised mouse yields a natural microenvironment for the skin, including vascularization, that cannot be mimicked in vitro.

For such experiments, male and female NOD/LtSscidIL2Rγₙᵤₗₗ mice are used, which are bred and maintained under specific pathogen-free conditions. Mice of this strain are virtually free of any T, B and NK cell activity and thus provide an optimal immunocompromised system for the grafting of human skin reconstructs without any rejection of the foreign tissue. All animals are provided with food and water ad libitum and they are housed in cages of maximally 5. The mice are used starting 6 weeks of age.

For grafting the reconstructs, a circular area of approximately 2 cm in diameter of mouse skin is excised under anesthesia leaving the carnosum intact. Mice are anaesthetized with Isoflurane (EZ aneasthesia machine). The graft is placed on the wound area and covered with dead mouse skin which is sutured with self-dissolving sutures. The mouse skin falls off within 14 days of suturing. The grafts do not pose discomfort to the mice. Mice are observed 5 times per week and are sacrificed by CO₂ inhalation if they show signs of discomfort. These signs include loss of body weight, bleeding from any orifice, listlessness, ruffled fur, reduced movement, as well as eruption of tumors through skin or the tumor exceeds 10% of body weight. Wounds heal within 4 to 8 weeks. Sick or distressed mice are euthanized in case of obvious signs of illness as listed above. Group size is between 5 and 10 animals, depending on preliminary experiments and the need for statistical analyses.

It is expected that animals receiving skin grafts or reconstruct containing dedifferentiated melanocytes or redifferentiated melanocytes as described herein will exhibit wound healing and/or repair of damaged tissue.

What is claimed is:

1. A method for producing a dedifferentiated melanocyte comprising contacting a melanocyte with an agent that activates Notch1 and selecting for a dedifferentiated melanocyte characterized as proliferating under stem cell growth conditions, exhibiting an increase in the expression of neural crest-related genes, and exhibiting a decrease in late pigmentation-related genes as compared to melanocytes not contacted with the agent.

2. The method of claim 1, wherein the agent is intracellular Notch1.

3. The method of claim 2, wherein the intracellular Notch1 comprises an isolated protein.

4. The method of claim 2, wherein the intracellular Notch1 comprises a nucleic acid molecule encoding intracellular Notch1.

5. The method of claim 1, wherein the agent is a soluble ligand of Notch1.

6. The method of claim 5, wherein the soluble ligand is Jagged1, Jagged2, Delta1 or Delta4.

7. The method of claim 6, wherein the soluble ligand comprises an isolated protein.

8. The method of claim 6, wherein the soluble ligand comprises a nucleic acid molecule encoding the soluble ligand.

9. The method of claim 1, wherein the agent is a Notch1 agonistic antibody.

10. The method of claim 1, wherein the agent is an inhibitory RNA molecule that blocks expression of an endogenous Notch1 inhibitor.

11. The method of claim 1, wherein the agent is a small organic molecule.

12. The method of claim 1, wherein the step of selecting for a dedifferentiated melanocyte comprises culturing the melanocyte in a stem cell medium.

13. The method of claim 1, wherein the neural crest-related genes comprise Msx1, Dlx1, Snail, Slug, Twist, p75, and SOX10.

14. The method of claim 1, wherein the late pigmentation-related genes comprise S100, TYRP1, MITF and HMB45.

15. The method of claim 1, further comprising the step of differentiating the dedifferentiated melanocyte into a neuronal cell, smooth muscle cell, oligodendrocyte, melanocyte, or chondrocyte.

16. A dedifferentiated melanocyte produced by the method of claim 1.

17. A pharmaceutical composition comprising the dedifferentiated melanocyte of claim 16.

18. A tissue reconstruct comprising a dedifferentiated melanocyte produced by the method of claim 1.

19. A tissue reconstruct comprising a neuronal cell, smooth muscle cell, oligodendrocyte, melanocyte, or chondrocyte produced by the method of claim 15.

20. A method for promoting tissue regeneration or repair comprising administering to a subject in need of treatment a pharmaceutical composition of claim 17 so that tissue regeneration or repair is promoted.

21. A method for promoting tissue regeneration or repair comprising administering to a subject in need of treatment a tissue construct of claim 18 so that tissue regeneration or repair is promoted.

22. A method for promoting tissue regeneration or repair comprising administering to a subject in need of treatment a tissue construct of claim 19 so that tissue regeneration or repair is promoted.

23. A method for identifying an agent that induces dedifferentiation of a melanocyte comprising contacting a melanocyte with a test agent and determining whether the test agent induces Notch1 signaling, wherein an induction in Notch1 signaling is indicative of an agent that induces dedifferentiation of a melanocyte.

24. A method for promoting tissue regeneration or repair comprising administering to a subject in need of treatment an agent identified by the method of claim 23 so that tissue regeneration or repair is promoted.


SEQUENCE LISTING (26 sequences)

SEQ ID NO: 1 - RNA, 19 nt, Artificial Sequence
  Synthetic oligonucleotide
gcaccugcccaguggaucc

SEQ ID NO: 2 - RNA, 19 nt, Artificial Sequence
  Synthetic oligonucleotide
guagcacauugcaacaaca

SEQ ID NO: 3 - DNA, 21 bp, Artificial Sequence
  Synthetic oligonucleotide
tggaaggatcgactcggtaaa

SEQ ID NO: 4 - DNA, 23 bp, Artificial Sequence
  Synthetic oligonucleotide
actgagtccaagatgacgaagct

SEQ ID NO: 5 - DNA, 21 bp, Artificial Sequence
  Synthetic oligonucleotide
cagtttgcagttgcaggcttt

SEQ ID NO: 6 - DNA, 21 bp, Artificial Sequence
  Synthetic oligonucleotide
tccggcagagctaggtactga

SEQ ID NO: 7 - DNA, 23 bp, Artificial Sequence
  Synthetic oligonucleotide
acctctttgctccctgagttcac

SEQ ID NO: 8 - DNA, 22 bp, Artificial Sequence
  Synthetic oligonucleotide
gactcttccagccactttttgg

SEQ ID NO: 9 - DNA, 18 bp, Artificial Sequence
  Synthetic oligonucleotide
accctgaacggatctacc

SEQ ID NO: 10 - DNA, 21 bp, Artificial Sequence
  Synthetic oligonucleotide
ctgcttgttgtcccggtagaa

SEQ ID NO: 11 - DNA, 20 bp, Artificial Sequence
  Synthetic oligonucleotide
ttcctcgccaagcacatttt

SEQ ID NO: 12 - DNA, 21 bp, Artificial Sequence
  Synthetic oligonucleotide
cctcccggtgacactaagtca

SEQ ID NO: 13 - DNA, 20 bp, Artificial Sequence
  Synthetic oligonucleotide
gactagagtctgagatgccc

SEQ ID NO: 14 - DNA, 21 bp, Artificial Sequence
  Synthetic oligonucleotide
cagacattgttaaattgcccg

SEQ ID NO: 15 - DNA, 21 bp, Artificial Sequence
  Synthetic oligonucleotide
tcgagagatgatgcaggacgt

SEQ ID NO: 16 - DNA, 20 bp, Artificial Sequence
  Synthetic oligonucleotide
tctggctcttcctcgctgtt

SEQ ID NO: 17 - DNA, 15 bp, Artificial Sequence
  Synthetic oligonucleotide
cctcgcccggctttg

SEQ ID NO: 18 - DNA, 21 bp, Artificial Sequence
  Synthetic oligonucleotide
gcctcgtagtctgcgttgaag

SEQ ID NO: 19 - DNA, 18 bp, Artificial Sequence
  Synthetic oligonucleotide
ctcgttaatacgcgctcg

SEQ ID NO: 20 - DNA, 20 bp, Artificial Sequence
  Synthetic oligonucleotide
aagtcctccaagccggtcat

SEQ ID NO: 21 - DNA, 20 bp, Artificial Sequence
  Synthetic oligonucleotide
acccgagatcctgcagatga

SEQ ID NO: 22 - DNA, 20 bp, Artificial Sequence
  Synthetic oligonucleotide
gccgtatgcagcattttcag

SEQ ID NO: 23 - DNA, 20 bp, Artificial Sequence
  Synthetic oligonucleotide
gtgcatgaacgaggtgaccc

SEQ ID NO: 24 - DNA, 20 bp, Artificial Sequence
  Synthetic oligonucleotide
gtattaacgccctcgcacgt

SEQ ID NO: 25 - DNA, 20 bp, Artificial Sequence
  Synthetic oligonucleotide
gttcgacagtcagccgcatc

SEQ ID NO: 26 - DNA, 19 bp, Artificial Sequence
  Synthetic oligonucleotide
ggaatttgccatgggtgga